(19) United States
(12) Patent Application Publication
(10) Pub. No.: US 2019/0274875 A1
(43) Pub. Date: Sep. 12, 2019

(54) THERMALLY-CONDUCTIVE BANDAGES AND WRAPS

(71) Applicant: James R. Bowers, Chapel HIll, NC (US)

(72) Inventor: James R. Bowers, Chapel HIll, NC (US)

(21) Appl. No.: 16/300,000

(22) Filed: Mar. 12, 2019

Related U.S. Application Data
(60) Provisional application No. 62/641,504, filed on Mar. 12, 2018.

(51) Int. Cl. A61F 7/02; A61F 5/01; A61F 13/00; A61F 7/08; A61F 7/00

(52) U.S. Cl. CPC A61F 7/02; A61F 2007/0035; A61F 5/0118; A61F 5/0111; A61F 13/00063; A61F 7/08; A61F 7/007; A61F 2005/0188; A61F 2007/0096; A61F 2007/0094; A61F 2007/0233; A61F 2007/0042; A61F 2007/0032; A61F 2007/0044; A61F 5/0109

(57) ABSTRACT

Provided herein are compositions and methods for providing a combination of compression and temperature therapy. In particular, provided herein are compression bandages that transmit cooling or heating to an injury.

### CROSS REFERENCE TO RELATED APPLICATIONS

The present application claims the priority benefit of U.S. Provisional Patent Application 62/641,504, filed Mar. 12, 2018, which is incorporated by reference in its entirety.

### FIELD OF THE INVENTION

Provided herein are compositions and methods for providing a combination of compression and temperature therapy. In particular, provided herein are compression bandages that transmit cooling or heating to an injury.

### BACKGROUND OF THE INVENTION

The most common sports injuries are sprains and strains of muscles and tendons. The most common treatment for these injuries is R.I.C.E. Therapy: Rest. Stop the activity immediately. Continued stress can increase the damage done to the tissue. Ice. Cold reduces the size of the blood vessels, thereby decreasing the bleeding, reducing swelling, and easing pain. Apply compression with a compression bandage. Compression helps to reduce swelling. Elevation. Elevate the injured limb above the level of the heart to help drain excess fluid from the area. Additionally, compression and cold therapy are standard protocols following surgery.

Existing compression wraps are constructed from insulating material. Thus, compression and ice must be applied separately. Improved compositions and methods for delivering a combination of compression and elevation are needed.

### SUMMARY OF THE INVENTION

Provided herein are compositions and methods for providing a combination of compression and temperature therapy. In particular, provided herein are compression bandages that transmit cooling or heating to an injury. The devices described herein provide an improvement in patient care over existing devices, which are made from insulating material and do not allow for simultaneous cooling and compression and/or support.

For example, in some embodiments, provided herein is a medical support device, wherein at least a portion of the medical support device is constructed of a thermally conductive material. In some embodiments, the medical support device is, for example, a compression bandage or wrap and/or a brace. In some embodiments, the compression bandage is an elastic bandage. In some embodiments, the brace is a brace configured to support a joint (e.g., an elbow, an ankle, or a wrist). In some embodiments, the thermally conductive material comprises a thermal conductor integrated into the material or the device or in a coating. In some embodiments, the thermal conductor comprises or is a metal. In some embodiments, the thermally conductive material comprises thermally conductive fabric. In some embodiments, all of the medical support device comprises a thermally conductive material. In some embodiments, the device further comprises a sensor configured to detect pressure, temperature, location, or movement of a body part. In some embodiments, the device comprises two conductive zones, separated by a non-conductive zone. Such an embodiment facilitates attachment of electrodes or sensors to the distinct conductive zones for delivery of electric current or monitoring (e.g., monitoring movement or location) the device and the underlying body part(s).

Further embodiments provide a system, comprising: a) a support device described herein; and b) a cooling component. In some embodiments, the system further comprises an electronic component configured to receive information from the sensor and display, story, or transmit the information. In some embodiments, the cooling component comprises a chemical cooling material or ice.

Additional embodiments provide a method of cooling a body part, comprising: contacting the body part with a device or system described herein under conditions such that the body part is cooled. In some embodiments, the body part is a joint or limb.

Additional embodiments are described herein.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a conductive fiber” is a reference to one or more conductive fibers and equivalents thereof known to those skilled in the art, and so forth.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of and/or ” consisting essentially of embodiments, which may alternatively be claimed or described using such language.

As used herein, the term “thermal transfer” refers to conveying thermal energy by one object to another.

As used herein, the term “thermally conductive” refers to the property of a material to transfer or pass thermal energy or heat to another material or through itself. Thus, a thermally conductive material readily transfers thermal energy, either by conduction, convection or radiation, to another material or through itself.

As used herein, the term “thermally insulating” refers to the inability of a material to transfer or pass thermal energy or heat to another material or through itself. Thus, a thermally non-conductive material does not readily transfer thermal energy to another material or through itself. A thermally insulating material is a thermally non-conductive material.

### DESCRIPTION OF THE INVENTION

Provided herein are compositions and methods for providing a combination of compression and temperature therapy. In particular, provided herein are compression bandages that transmit cooling or heating to an injury. The devices herein, find use in a variety of applications, for example reducing pain and swelling, and speeding recovery from injuries (e.g., soft tissue injury, sprain, broken bone, etc.) and/or surgeries.

The present disclosure provides medical support devices comprising conductive materials. The conductive material allows for a cooling component placed on the outside of the device to provide cooling of the body part without removing the device. The support devices allow for improved methods of simultaneously cooling and providing compression and/or support to aid in recovery from injury and surgery or for other uses. This results in faster recovery from injury and surgery.

In some embodiments, devices herein comprise a material with sufficient conductive capacity (e.g., comprising sufficient ratio of conductive to non-conductive sub-materials (e.g., 10:1, 9:1, 8:1, 7:1, 6:1, 5:1, 4:1, 3:1, 2:1, 1:1, 1:2, 1:3, 1:4, 1:5, 1:6, 1:7: 1:8, 1:9, 1:10, or ranges therebetween, depending upon the conductive and isolative capacity of the materials)) to render the material as a whole conductive. For example, in some embodiments, devices or materials that comprise metallic components (e.g., metal threads, wires, electronics, etc.), but do not comprise a sufficient amount of conductive material to render the device or overall material conductive, are not within the scope herein.

The present disclosure is not limited to particular medical support devices. Examples include, but are not limited to, braces, wraps, bandages, and the like. In some embodiments, the support device is an elastic wrap or bandage. In some embodiments, the brace is configured (e.g., sized and shaped) to provide support to a limb or joint (e.g., knee, elbow, wrist, angle, etc.). In some embodiments, a device is a sleeve, legging, or sock.

The present disclosure is not limited to particular thermally conductive material. In some embodiments, a thermal conductor (e.g., metal) is incorporated into material (e.g., woven into) used to construct a support device. In some embodiments, conductive threads are woven into a material to impart conductivity to the material. For example, in some embodiments, conductive fabrics are utilized (e.g., commercially available from V Technical Textiles Inc., Palmyra, N.Y. or Eeonyx, Pinole, Calif.). In some embodiments, a conductive coating is placed on the outside of the device. In some embodiments, a device comprises a thermally conductive material with a thermal conductivity of 1 watt/mK or greater (e.g., 1 watt/mK, 2 watt/mK, 5 watt/mK, 10 watt/mK, 20 watt/mK, 30 watt/mK, 40 watt/mK, 50 watt/mK, 100 watt/mK, or greater, or ranges therebetween (e.g., 10 watt/mK or greater)). The materials (e.g., conductive and insulating) have a net composite conductivity that is 1 watt/mK or greater (e.g., 1 watt/mK, 2 watt/mK, 5 watt/mK, 10 watt/mK, 20 watt/mK, 30 watt/mK, 40 watt/mK, 50 watt/mK, 100 watt/mK, or greater, or ranges therebetween (e.g., 10 watt/mK or greater)).

In some embodiments, devices herein comprise one or more conductive materials (e.g., woven together with other materials (e.g., elastic materials, insulating materials, etc.). Suitable conductive materials may comprise metals, including, but not limited to: aluminum, cadmium, chromium, cobalt, copper, iron, manganese, nickel, platinum, palladium, rhodium, silver, gold, tin, titanium, tungsten, vanadium, zinc, etc., and suitable alloys thereof, such as alloys of aluminium (e.g., Al—Li, alumel, duralumin, magnox, zamak, etc.), alloys of iron (e.g., steel, stainless steel, surgical stainless steel, silicon steel, tool steel, cast iron, Spiegeleisen, etc.), alloys of cobalt (e.g., stellite, talonite, etc.), alloys of nickel (e.g., German silver, chromel, mu-metal, monel metal, nichrome, nicrosil, nisil, nitinol, etc.), alloys of copper (beryllium copper, billon, brass, bronze, phosphor bronze, constantan, cupronickel, bell metal, Devarda's alloy, gilding metal, nickel silver, nordic gold, prince's metal, tumbaga, etc.), alloys of silver (e.g., sterling silver, etc.), alloys of tin (e.g., Britannium, pewter, solder, etc.), alloys of gold (electrum, white gold, etc.), amalgam, etc. In some embodiments, a device material comprises 1-75% conductive material (e.g., 1%, 2%, 3%, 4%, 5%, 10%, 15%, 20%, 25%, 30%, 40%, 50%, 60%, 70%, 75%, or ranges therebetween).

In some embodiments, devices herein comprise one or more insulating materials. In some embodiments, devices comprise one or more fibrous materials. Suitable fibrous materials may be natural (e.g., cotton, wool, animal fiber, etc.) or synthetic (e.g., polyester, rayon, etc.). In some embodiments, device material comprises 25-99% insulating and/or fibrous material (e.g., 25%, 30%, 40%, 50%, 60%, 70%, 75%, 80%, 85%, 90%, 95%, 96%, 97%, 98%, 99%, or ranges therebetween). In some embodiments, insulating and/or fibrous materials that find use in materials herein (e.g., along with conductive materials) include, but are not limited to a fiber, trace, yarn, strand, or textile selected from the group consisting of: acetate, acrylic, cotton, denim, elastane, Kevlar, latex, linen, Lycra, neoprene, nylon, nylon, polyester, wool, silicon rubber, silk, spandex, Danconn, rayon, etc.

In some embodiments, a portion (e.g., specific sections or all) of the device comprises the conductive material. In some embodiments, it may be advantageous to provide portions of the device that do not comprise conductive material (e.g., straps, closures, support elements, etc.).

In some embodiments, devices further comprises sensors (e.g., to measure temperature, pressure, etc.) to aid in determining if an injury is healing or if a support device needs adjusting. In some embodiments, sensors are placed in regions of the devices that are not conductive. In some embodiments, sensors are placed in regions of the devices that are conductive (e.g., separated by non-conductive portions). In some embodiments, sensors interact wirelessly or over wires with electronic devices (e.g., smart phones, smart watches, or computers) that collect, transmit, analyze, or display data.

In some embodiments, devices are provided in a system with a source of cooling (e.g., chemical cooling packs such as those commercially available from Dynarex, Orangeburg, N.Y.). The devices described herein can be used with any source of cooling (e.g., ice or freezable chemical cold packs).

The devices described herein find use in cooling limbs, joints, or other body parts in any numbers of situations. Examples include, but are not limited to, after injury (e.g., sports injury), accidents, surgery, illness, etc.

In some embodiments, provided herein are thermally-conductive compression bandages having any suitable dimensions. In some embodiments, a bandage (or any device herein) is 1-10 mm in thickness (e.g., 1 mm, 2 mm, 3 mm, 4 mm, 5 mm, 6 mm, 7 mm, 8 mm, 9 mm, 10 mm, or ranges therebetween). In some embodiments, a bandage is 2-20 cm in width (e.g., 2 cm, 3 cm, 4 cm, 5 cm, 6 cm, 7 cm, 8 cm, 9 cm, 10 cm, 11 cm, 12 cm, 13 cm, 14 cm, 15 cm, 16 cm, 17 cm, 18 cm, 19 cm, 20 cm, or ranges therebetween). In some embodiments, a bandage is 10 cm to 3 meters in length (e.g., 10 cm, 15 cm, 20 cm, 30 cm, 40 cm, 50 cm, 75 cm, 1 m, 1.25 m, 1.5 m, 1.75 m, 2 m, 2.25 m, 2.5 m, 2.75 m, 3 m, or ranges therebetween).

In some embodiments, a bandage or other device herein comprises a material with an elastic modulus of 0.02 to 0.2 N/mm (e.g., 0.02 N/mm, 0.03 N/mm, 0.04 N/mm, 0.05 N/mm, 0.06 N/mm, 0.07 N/mm, 0.08 N/mm, 0.09 N/mm, 0.1 N/mm, 0.11 N/mm, 0.12 N/mm, 0.13 N/mm, 0.14 N/mm, 0.15 N/mm, 0.16 N/mm, 0.17 N/mm, 0.18 N/mm, 0.19 N/mm, 0. 2 N/mm, or ranges therebetween).

In some embodiments, a bandage or other device herein comprises a material with an elongation at 10 cN/cm of 120-300% (e.g., 120%, 130%, 140%, 150%, 160%, 170%, 180%, 190%, 200%, 210%, 220%, 230%, 240%, 250%, 260%, 270%, 280%, 290%, 300%, or ranges therebetween).

All publications and patents mentioned in the above specification are herein incorporated by reference as if expressly set forth herein. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in relevant fields are intended to be within the scope of the following claims.

We claim:

1. A medical device comprising an elastic material wherein at least a portion of said elastic material comprises thermally conductive material.

2. The device of claim 1, wherein said medical device is selected from the group consisting of a compression bandage, a wrap, a brace, a sleeve, a legging, or a sock.

3. The device of claim 2, wherein said compression bandage is an elastic bandage.

4. The device of claim 2, wherein said brace is a brace configured to support a joint.

5. The device of claim 4, wherein said joint is a knee joint, an elbow, an ankle, or a wrist.

6. The device of claim 1, wherein said thermally conductive material comprises a thermal conductor integrated into the material or the device or in a coating.

7. The device of claim 6, wherein said thermal conductor comprises a metal.

8. The device of claim 6, wherein said thermally conductive material comprises thermally conductive fabric.

9. The device of claim 6, wherein all of said medical device comprises a thermally conductive material.

10. The device of claim 1, wherein said device further comprises a sensor configured to detect pressure or temperature of a body part.

11. A system, comprising:
a) the device of claim 1; and
b) a cooling component.

12. The system of claim 11, wherein said system further comprises an electronic component configured to receive information from said sensor and display or transmit said information.

13. The system of claim 11, wherein said cooling component comprises a chemical cooling material or ice.

14. A method of cooling a body part, comprising:
contacting said body part with the system of claim 11 under conditions such that said body part is cooled.

13. The method of claim 12, wherein said body part is a joint or limb.
